(19) United States
(12) Patent Application Publication
(10) Pub. No.: US 2020/0140251 A1
(43) Pub. Date: May 7, 2020

(54) PUNCTURING ELEMENT FOR GRADUAL LIQUID FLOW

(71) Applicant: NOVAMED LTD., Jerusalem (IL)

(72) Inventors: Emil KATZ, Savyon (IL); Gadi PORATH, Kiryat Tivon (IL)

(21) Appl. No.: 16/619,917

(22) PCT Filed: Jun. 7, 2018
(86) PCT No.: PCT/IL2018/050625

§ 371 Date: Dec. 5, 2019

(30) Foreign Application Priority Data
Jun. 7, 2017 (IL) 252764

(51) Int. Cl. B67B 7/00; B01L 3/00

(52) U.S. Cl. CPC B67B 7/24; A61B 10/0045; B01L 3/502

(57) ABSTRACT

The present invention relates to a puncturing element which allows gradual puncturing of an openable liquid seal of a relatively small liquid filled chamber, thereby facilitating gradual pouring out and mixing of the liquid from the chamber.

### FIELD OF THE INVENTION

The present invention relates to a puncturing element for puncturing a puncturable surface. More particularly, the invention relates to a puncturing element which allows gradual puncturing of an openable liquid seal of a relatively small liquid filled chamber (e.g., a chamber having a diameter of only few millimeters), thereby facilitating gradual pouring out and mixing of the liquid from the chamber. The invention further relates to an apparatus comprising the puncturing element. In some embodiments, the apparatus is an assay device for determining the presence and/or amount of an analyte of interest in a sample.

### BACKGROUND OF THE INVENTION

Laboratory, medical or industrial assays for qualitatively assessing or quantitatively measuring the presence or amount or the functional activity of a target entity (the analyte) in a sample usually contain one or more reagents.

The reagents may be liquid reagents appropriate for the assay in question. Often, the reagents need to be mixed adjacent to their interaction with the sample. Therefore, the reagents are maintained within separate liquid chambers, sealed with openable liquid seals. Upon activation, the seals need to be punctured for allowing the one or more liquid reagents to flow out, be mixed and come into contact with the sample.

Thus there is need for a puncturing element which is capable of making a hole in the chamber, without it blocking the punctured hole, and which enables the liquid to pour out in a manner that ensures the mixing of the one or more reagents.

It is an object of the present invention to provide a puncturing element for the gradual puncturing of a liquid filled chamber thereby facilitating gradual pouring out of the liquid from the chamber.

Other objects and advantages of the invention will become apparent as the description proceeds.

### SUMMARY OF THE INVENTION

The present invention relates to a puncturing element for enabling a gradual liquid flow, comprising a puncturing surface having a formation adapted to ensure a gradual puncturing of a puncturable surface (e.g., of a liquid filled chamber).

According to an embodiment of the invention, the puncturing element further comprises a base portion for supporting the puncturing surface.

According to an embodiment of the invention, the puncturing surface is disposed at an angle relative to the puncturable surface. The angle of the puncturing surface relative to the puncturable surface ensures the gradual puncturing of the puncturable surface.

According to an embodiment of the invention, the puncturing surface comprises at least one jagged tooth. According to some embodiments, the jagged tooth has a sharp tip, so that it is capable of cutting (thereby puncturing) the puncturable surface upon contact. The arrangement of the at least one jagged tooth on the puncturing surface allows gradual cutting of the puncturable surface.

According to an embodiment of the invention, the base portion of the puncturing element is adapted to be attached or secured to an apparatus that as part of its operation, the apparatus is configured to release liquid from a liquid filled chamber that is sealed by the puncturable surface.

According to an embodiment of the invention, the puncturing surface comprises jagged teeth arranged differently on said puncturing surface.

According to an embodiment of the invention, the jagged teeth are arranged in either a uniform spaced pattern or in a non-uniform pattern. For example, the patterns may include a circular or row-by-row arrangement.

According to an embodiment of the invention, the height of each tooth is vary, so that one or more of the teeth are shorter or longer than others to allow gradual cutting or puncturing of the puncturable surface.

According to an embodiment of the invention, the jagged teeth have essentially the same length, but they are being ordered in an inclined manner along the puncturable surface as to allow gradual cutting or puncturing of the puncturable surface.

In another aspect, the present invention further relates to an apparatus comprising:
a) at least one puncturing element that comprises a puncturing surface having a formation adapted to ensure a gradual puncturing of a puncturable surface; and
b) at least one liquid filled chamber which is sealed by an openable liquid seal.

According to an embodiment of the invention, the apparatus further comprises a mixing chamber. The base portion of the puncturing element is coupled to an inner surface of the apparatus that is disposed at an angle relative to the mixing chamber.

According to an embodiment of the invention, the puncturing surface of the puncturing element is disposed at an angle relative to the openable liquid seal of the liquid filled chamber.

According to an embodiment of the invention, the puncturing element is capable of puncturing the openable liquid seal of the at least one liquid filled chamber, thereby facilitating gradual pouring out and mixing of the liquids.

The gradual puncturing of the puncturable surfaces of the liquid filled chambers and consequently, the gradual pouring out of the liquids by gravitational force, onto an inner surface of an apparatus having an inclined surface causes mixing of the one or more liquids prior to their flow into the mixing chamber.

According to an embodiment of the invention, the apparatus is an assay device for determining the presence and/or amount of an analyte of interest in a sample.

### BRIEF DESCRIPTION OF THE DRAWINGS

In the drawings:

FIG. 1 schematically illustrates a perspective view of a puncturing element, according to an embodiment of the invention;

FIG. 2A schematically illustrates a cross-sectional view of an assay device, prior to the actuation of its actuating lid, according to an embodiment of the invention;

FIG. 2B schematically illustrates a cross-sectional view of the assay device of FIG. 2B, after the actuation of its actuating lid, according to an embodiment of the invention;

FIG. 3 schematically illustrates a semi-exploded view of the assay device of FIG. 2A according to an embodiment of the present invention; and

FIG. 4 schematically illustrates the actuation of the actuating lid of the assay device of FIG. 3.

### DETAILED DESCRIPTION OF THE INVENTION

Reference will now be made to several embodiments of the present invention, examples of which are illustrated in the accompanying figures. Wherever practicable similar or like reference numbers may be used in the figures and may indicate similar or like functionality. The figures depict embodiments of the present invention for purposes of illustration only. One skilled in the art will readily recognize from the following description that alternative embodiments of the structures and methods illustrated herein may be employed without departing from the principles of the invention described herein.

A puncturing element for puncturing a puncturable surface is described herein. In some embodiments, the puncturable surface is an openable seal. In some embodiments, the puncturing element allows gradual puncturing of an openable liquid seal of a liquid filled chamber or a capsule thereby facilitating gradual pouring out of the liquid from the chamber or the capsule.

In some embodiments, the liquid filled chamber is a relatively small chamber which comprises a liquid volume of between 0.5 ml and 1.5 ml.

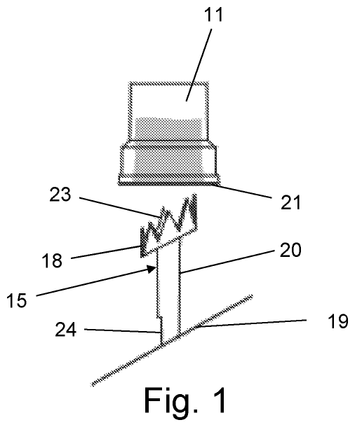

Referring now to FIG. 1, in some embodiments, the puncturing element 15 includes a base portion 20 and a puncturing surface 18. The base portion 20 is adapted to support the puncturing surface 18. For example, base portion 20 can be provided in form of an elongated support portion for the puncturing portion 18. The puncturing surface 18 is disposed at an angle relative to a puncturable surface 21 of a liquid filled chamber or capsule. The puncturing surface 18 may include at least one jagged tooth 23.

In some embodiments, the at least one jagged tooth 23 is sharp at its tip so that it is capable of cutting (thereby puncturing) the puncturable surface 21 upon contact.

According to some embodiments, jagged teeth (i.e., a plurality of jagged tooth 23) may be arranged differently on the puncturing surface 18. The teeth may be arranged in either a uniform spaced pattern or in a non-uniform pattern. The patterns include, but are not limited to, a circular or row-by-row arrangement. The height of each tooth may also vary, in other words, some of the teeth may be shorter or longer than others. The different arrangements of the at least one jagged tooth 23 on the puncturing surface 18 allow gradual cutting (puncturing) of the puncturable surface 21.

For example, puncturable surface 21 can be provided in form of an openable liquid seal that is manufactured from a material that can be punctured to release the liquid contained within liquid filled chamber 11.

The angle of the puncturing surface 18 relative to the puncturable surface 21 and the at least one jagged tooth 23 on the puncturing surface 18 ensure the gradual pouring out of liquid once the openable seal of a liquid filled chamber 11 is punctured by the puncturing element 15.

In some embodiments, the base portion 20 of the puncturing element 15 includes a notch 24 or other attaching means suitable for securing the puncturing element 15 to an inner surface 19 of an apparatus, machine or device that as part of its operation it is configured to release liquid from a sealed chamber or capsule, e.g., such as an assay device that is configured for detection of one or more analytes in a sample by releasing a specific regent from a sealed chamber.

In some embodiments, the base portion 20 of the puncturing element 15 is fixedly coupled to the inner surface 19, or it can be implemented as an integral part of such apparatus, machine or device.

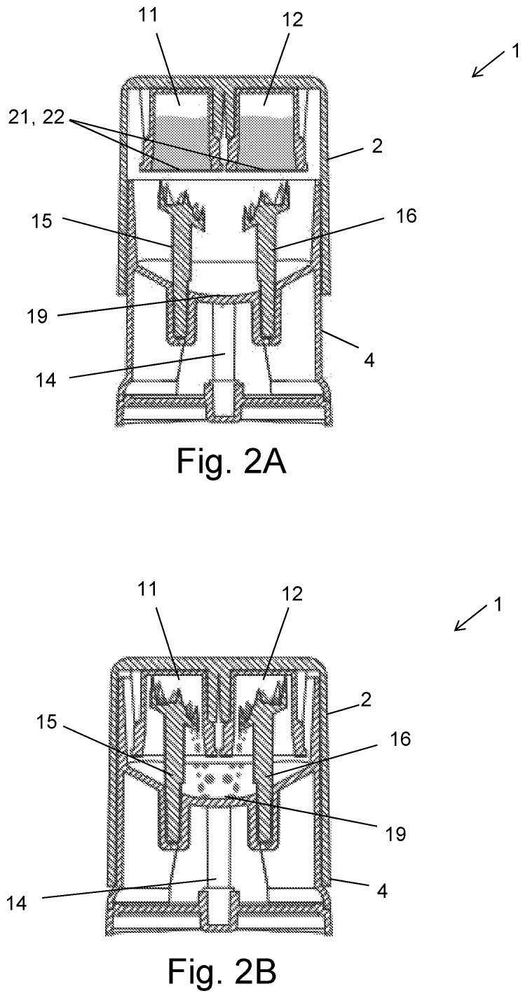

In some embodiments, the base portion 20 of the puncturing element 15 is coupled to inner surface 19 of an apparatus, disposed at an angle relative to a mixing chamber (e.g., as indicated by numeral 14 in FIGS. 2A and 2B) of the apparatus, therefore having an inclined surface. For example, puncturing of an openable liquid seal 21 of a liquid filled chamber 11 facilitates gradual pouring out of the liquid from the chamber 11, by gravitational force, and flowing of the liquid onto the inclined surface of the inner surface 19 of the apparatus, thereby enabling the mixing of the liquid prior to its flow into the mixing chamber, where it is contacted with a sampling device.

In some embodiments, the invention provides a puncturing element 15 for liquid filled chamber 11 wherein the liquid filled chamber 11 is sealed with openable liquid seal 21 (i.e., the puncturable surface). The puncturing surface 18 of the puncturing element 15 faces the openable liquid seal 21 of the liquid filled chamber 11 and is disposed at an angle relative to the surface of the openable liquid seal 21. The puncturing surface 18 includes at least one jagged tooth 23, such that the at least one jagged tooth 23 is disposed at an angle relative to the puncturable surface of the openable liquid seal 21. The at least one jagged tooth 23 is sharp at its tip so that it is capable of cutting (thereby puncturing) the openable liquid seal 21 upon contact. The arrangement of the at least one jagged tooth 23 on the puncturing surface 18 allows gradual cutting of the openable liquid seal 21 upon contact.

For example, upon applying external force, the puncturing surface 18 of the puncturing element 15 is pushed through (i.e. punctures) the openable liquid seal 21 of the liquid filled chamber 11, allowing gradual pouring of the liquid content onto inner surface 19, by gravitational force. The sharp end of at least one jagged tooth 23 on the puncturing surface 18 of the puncturing element 15 ensures that the openable liquid seal 21 is punctured. The angle of the puncturing surface 18 relative to the surface of the openable liquid seal 21 and the arrangement of the at least one jagged tooth 23 allow the gradual cutting (puncturing) of the openable liquid seal 21 and therefore facilitates the gradual pouring out of the liquid contained in the chamber 11, by gravitational force.

In some embodiments, at least one puncturing element 15 is provided as part of an apparatus. In some embodiments, the apparatus is an assay device for determining the presence and/or amount of an analyte of interest in a sample.

FIGS. 2A and 2B schematically illustrate a cross-sectional view of an assay device 1 provided with puncturing elements, according to an embodiment of the present invention. Referring now to FIG. 2A, in some embodiments of the invention, assay device 1 comprises a first liquid filled chamber 11, a second liquid filled chamber 12, a first puncturing element 15, a second puncturing element 16, an inner surface of an apparatus 19, an actuating lid 2 and a housing 4 that serves to house one or more components of assay device 1. Each of the first and second liquid filled chambers 11, 12 may contain a liquid which is sealed therein by a corresponding first and second openable liquid seal 21, 22, respectively. For example, in this embodiment, the openable liquid seals 21 and 22 are located at the bottom end of each chamber 11, 12. The first chamber 11 may contain a first liquid and the second chamber 12 may contain a second liquid. Alternatively, both chambers 11 and 12 may contain the same content.

In some embodiments, the assay device 1 also comprises a mixing chamber 14 for receiving a mixed solution of the first and second liquids that were released from chambers 11, 12.

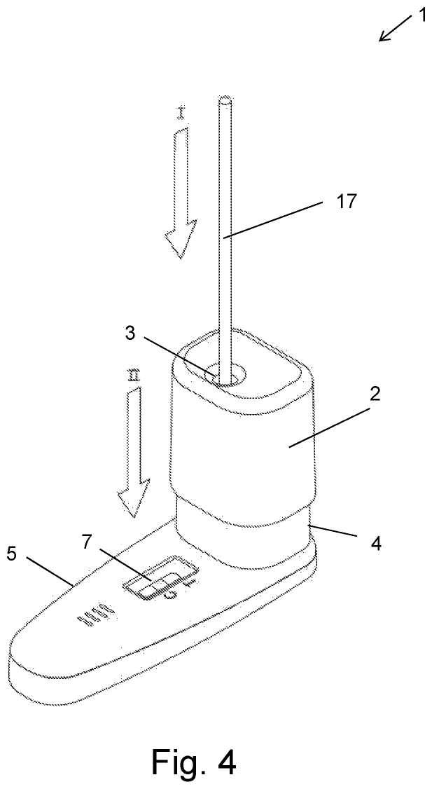

In order to ensure mixing of the liquids from both chambers 11, 12 an inner surface 19 at the interior of housing 4 is disposed at an angle relative to the mixing chamber 14 of the apparatus, therefore having an inclined surface and/or a spiral-like form that forces the liquids to flow, by gravitational force, e.g., in a spiral or twisted manner, towards the mixing chamber 14. The puncturing of the openable liquid seals 21, 22 of the liquid filled chambers 11, 12 facilitates gradual pouring out of each of the liquids from the chambers 11, 12, by gravitational force, and flow of the liquids onto the inclined surface of the inner surface 19. The inclined surface enables the mixing of the liquids prior to their flow into mixing chamber 14 (as shown in FIG. 2B), where they may contact with a sampling device (as indicated by numeral 17 in FIG. 4).

The liquid of the present invention may be aqueous in nature. Although the present invention is described herein with reference to liquids, it will be appreciated that the present invention is equally applicable for fluids.

The liquid may be a reagent appropriate for the assay in question. Examples of reagents may be chosen from, but not limited to, a binding reagent capable of binding to an analyte of interest, an enzyme, a surfactant, a buffer, an extraction reagent, a salt, a precipitation reagent, a viscosity modifying reagent and a lysing reagent. The binding reagent may be labelled with a detectable label. The one or more reagents may be provided within the liquid chambers. The reagents may be provided in the dry state or in the wet state.

Each of the openable liquid seals 21, 22 refers to a seal which is capable of being gradually removed or punctured in order to enable the liquid within the first and second chambers 11, 12 to flow onto the inner surface 19. The openable liquid seal 21, 22 may be puncturable. In this embodiment, the lid 2 is capable of being actuated to cause the openable liquid seals 21, 22 to be punctured or removed, allowing the liquid content to pour out, by gravitational force, onto the inner surface 19.

The openable liquid seals 21, 22 may be chosen from a material having low fluid permeability such as aluminum or other metal, alloy or polymer layer. The layer may have a thickness in the range of from about 15 to about 100 microns. The layer may further comprise a bondable backing layer such as a lacquer or a laminate to enable the layer to be heat sealed or bonded to the liquid chambers.

In some embodiments the openable liquid seals 21, 22 are laminated aluminum heat sealed seals having a thickness of 0.08 mm.

In some embodiments, the actuation of lid 2 is obtained by a pushable mechanism that enables to press/push the lid 2 toward the housing 4 of device 1, thereby causing the openable liquid seals 21, 22 to be gradually punctured or removed by the first puncturing element 15 and second puncturing element 16, respectively, which are located within the interior of housing 4, with respect to the location of the sealed opening of chambers 11, 12. In other words, lid 2 may have two possible positions an initial position (i.e., pre-press) and a pressing position which causes the puncturing of the openable liquid seals 21, 22. A puncture force is needed in order to actuate lid 2 and to puncture the openable liquid seals 21, 22. For example, in FIG. 2B, after the actuating lid 2 has been pushed, the first puncturing element 15 and second puncturing element 16, are pushed through the openable liquid seals 21, 22 causing the gradual pouring of the liquid of the first and second chambers 11, 12 onto the inner surface 19 of the assay device 1, by gravitational force, thereby enabling the mixing of the liquids prior to their flow into mixing chamber 14, where they are contacted with the sampling device 17.

In some embodiments, assay device 1 may comprise at least one liquid filled chamber and at least one corresponding puncturing element. In case assay device 1 comprises a plurality of liquid filled chambers (such as chambers 11 and 12), each liquid filled chamber may contain a different content or at least some of the liquid chambers may contain the same liquid content. Where pluralities of liquid filled chambers are provided, the liquids may be maintained in each chamber and separated from each other by the openable liquid seal of each chamber. Thus the actuation of the lid 2, results in the gradual cutting of the openable liquid seal of each chamber (e.g., such as seals 21, 22) by the corresponding puncturing elements (e.g., such as elements 15, 16) facilitating the gradual pouring out of liquid from the liquid filled chambers onto the inner surface 19, where the liquids are mixed and flow into the mixing chamber 14, where they are contacted with the sampling device 17.

In some embodiments, the assay device 1 comprises a delay mechanism for delaying the interaction of the sample with the solution within the mixing chamber 14. The delay mechanism can be a sponge, a suitable orifice or any other mechanism or element capable of delaying the flow of the mixed solution to access the mixing chamber 14. The delay mechanism can be provided as a buffer at an opening of the mixing chamber 14.

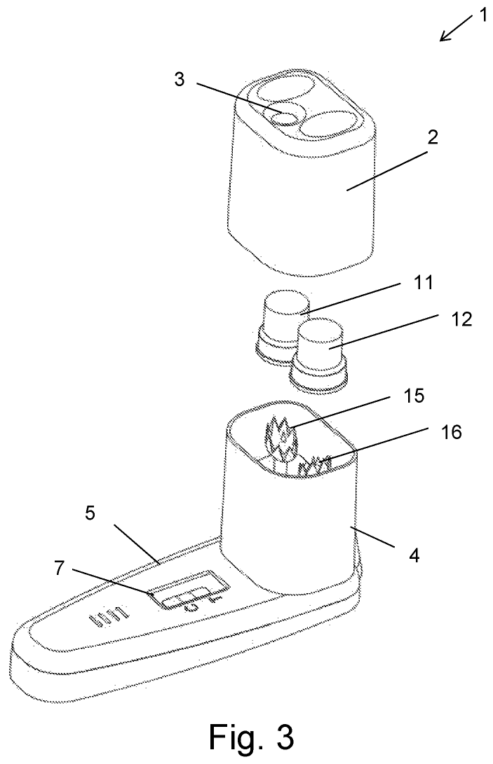

Referring now to FIG. 3, in this embodiment, the actuating lid 2 comprises the first and second chambers 11, 12 and an opening access 3 for enabling a sampling device (e.g. a swab) 17 to be inserted into the mixing chamber 14.

In some embodiments, in the initial position (i.e., pre-press), opening 3 may be blocked in order to prevent the insertion of the sampling device prior to the mixture of the liquids. Opening 3 may be unblocked manually (e.g., by removable of a sealing layer on top of it), or may be unblocked automatically during actuation of lid 2 (e.g., by automatically breaking or removing a sealing layer).

The mixing chamber 14 is also adapted to receive a sampling device 17 into the liquid mixing chamber 14 (e.g., by using a sample swab as shown in FIG. 4).

In some embodiments, assay device 1 may comprise assay means 5 (e.g., lateral flow assay arrangement). The assay means 5 may comprise a liquid pathway such as a capillary channel, a microfluidic pathway, or a porous flow through carrier such as a lateral flow porous carrier. The liquid pathway may lead to a detection chamber or zone 7. The porous flow through carrier may comprise one or a plurality of porous carrier materials which in use are fluidically connected. The plurality of porous carrier materials may be the same or different. The plurality of porous carriers may at least partially overlap one another in a linear or stacked arrangement. The assay means 5 may comprise a lateral flow carrier material. The assay means 5 may comprise a plurality of liquid pathways each defining a separate flow path for the detection of an analyte. The analyte may be the same or different. Where a plurality of liquid pathways are provided they may have a common sample receiving portion such that liquid from the mixing chamber 14 is able to flow to each flow channel.

As will be appreciated by a person skilled in the art, the assay means 5 can be implemented using a variety of detection methods. For example, the assay means 5 may comprise one or more reagents appropriate for the assay in question. The one or more reagents may be chosen from a binding agent capable of binding to the analyte of interest, a reagent that is able to react with the analyte of interest, such as an enzyme, a reagent that is able to interact or otherwise react with the products of any interaction between the analyte of interest and a further reagent. The assay means 5 may comprise a detection zone 7 which is capable of detecting a product of the interaction or reaction of the analyte of interest and one or more reagents. Other methods can also be used such as immobilising a labelled binding reagent for an analyte of interest, the use of an enzyme for the analyte of interest, the use of an electron mediator for the enzyme or a colour developing reagent and/or a precipitation reagent, etc.

The analyte of interest to be determined by the assay device may be of a biological, industrial or environmental nature. The analyte may be of a mammalian, especially of a human origin. The analyte of interest may be any of significance including toxins, organic compounds, proteins, peptides, microorganisms, bacteria, viruses, amino acids, nucleic acids, carbohydrates, hormones, steroids, vitamins and drugs. The analyte may be one which requires a liquid pre-treatment step before being exposed to an assay means. The liquid treatment step may comprise one or more of, but not limited to, a dilution, a liquid suspension, an extraction, a binding reaction, a biochemical reaction, a chemical reaction, a buffering, a treatment with a surfactant. The pre-treatment step may be carried out by introducing the analyte of interest into the liquid container and allowing it to interact with the liquid container therein. The liquid container may comprise one or more reagents which enable a pre-treatment step to be carried out. In particular, analytes of interest include Streptococcus A, Candida organisms and bacterial vaginosis organisms.

The sample can be derived from any source, such as a physiological liquid, including blood, serum, plasma, saliva, sputum, ocular lens liquid, sweat, urine, milk, ascites liquid, mucous, synovial liquid, peritoneal liquid, transdermal exudates, pharyngeal exudates, bronchoalveolar lavage, tracheal aspirations, cerebrospinal liquid, semen, cervical mucus, vaginal or urethral secretions, amniotic liquid, and the like.

Referring now to FIG. 4, a user may simply and easily insert a sampling device 17 (i.e., such as swab) into the mixing chamber 14 via opening 3, either prior to or after actuation of lid 2 (indicated by step I in the figure). After actuating lid 2 has been pushed (indicated by step II in the figure), the first puncturing element 15 and second puncturing element 16, puncture the openable liquid seals 21, 22, exerted by the cutting of the at least one jagged tooth of the puncturing surface of each puncturing element 15 and 16, thereby causing the gradual pouring out of the liquid from the first and second chambers 11, 12 onto the inner surface 19, followed by their flow into the mixing chamber 14. Thus the need to use excessive force in order to contact the sample with the liquid provided within the mixing chamber 14 is minimized.

The actuation of lid 2 may result in mixing of the liquid contained in the respective liquid chambers 11, 12 (e.g., mixing of a liquid in one liquid chamber 11 with a liquid in another liquid chamber 12). In order to ensure the mixing of the liquids from both chambers 11, 12 an inner surface 19, at the interior of housing 4, is disposed at an angle relative to the mixing chamber 14 of the apparatus, therefore having an inclined surface and/or a spiral-like form that forces the liquids to flow in spiral or twisted manner towards the mixing chamber 14. Such a spiral flow manner may facilitate the mixing of both liquids.

All the above description and examples have been given for the purpose of illustration and are not intended to limit the invention in any way. Many different mechanisms and methods of analysis, can be employed, all without exceeding the scope of the invention.

1. A puncturing element, comprising a puncturing surface having a formation adapted to ensure a gradual puncturing of a puncturable surface, said puncturing surface comprising at least one jagged tooth ordered in an inclined manner, and wherein said puncturing surface is disposed at an angle relative to the puncturable surface to facilitate gradual pouring of liquid by gravitational force.

2. A puncturing element, according to claim 1, further comprising a base portion adapted to support the puncturing surface.

3. The puncturing element according to claim 1, wherein the at least one jagged tooth has a sharp tip.

4. The puncturing element according to claim 2, wherein the base portion is adapted to be attached or secured to an apparatus.

5. The puncturing element according to claim 1, wherein the puncturing surface comprises jagged teeth arranged differently on said puncturing surface.

6. The puncturing element according to claim 5, wherein the jagged teeth are arranged in either a uniform spaced pattern or in a non-uniform pattern.

7. The puncturing element according to claim 6, wherein the patterns include a circular or row-by-row arrangement.

8. The puncturing element according to claim 5, wherein the height of each tooth is varied, so that one or more of the teeth are shorter or longer than others to allow gradual cutting or puncturing of the puncturable surface.

9. The puncturing element according to claim 8, wherein each of the jagged teeth has the same length, while they are being ordered in an inclined manner as to allow gradual cutting or puncturing of the puncturable surface.

10. An apparatus, comprising:
a) at least one puncturing element having a formation adapted to ensure a gradual puncturing of a puncturable surface according to claim 1; and
b) at least one liquid filled chamber this is sealed with an openable liquid seal, wherein the at least one puncturing element allows gradual puncturing of the openable liquid seal as to facilitate gradual pouring out of liquid contained in said chamber by gravitational force.

11. The apparatus according to claim 10, wherein the at least one liquid filled chamber comprises a liquid volume of between 0.5 ml and 1.5 ml.

12. The apparatus according to claim 10, wherein the openable liquid seal is a laminated aluminum heat sealed seal having a thickness of 0.08 mm.

13. The apparatus according to claim 10, further comprises a mixing chamber of wherein the base portion of the at least one puncturing element is coupled to an inner surface of the apparatus this is disposed at an angle relative to the mixing chamber.

14. The apparatus according to claim 10, wherein the puncturing surface of the at least one puncturing element is disposed at an angle relative to the openable liquid seal of the at least one liquid filled chamber.

15. The apparatus according to claim 10, wherein the at least one puncturing element is capable of puncturing the openable liquid seal of the at least one liquid filled chamber, thereby facilitating gradual pouring out and mixing of the liquid.

16. The apparatus according to claim 10, wherein the apparatus is an assay device for determining the presence and/or amount of an analyte of interest in a sample.

17. (canceled)

18. (canceled)
